(19) United States
(12) Patent Application Publication
(10) Pub. No.: US 2015/0196350 A1
(43) Pub. Date: Jul. 16, 2015

(54) ELECTROSURGICAL DEVICES HAVING ENHANCED EFFECTIVENESS AND METHODS OF MAKING AND USING SAME

(71) Applicant: ElectroMedical Associates LLC, Bethesda, MD (US)

(72) Inventors: Yuval CARMEL, Rockville, MD (US); Anatoly SHKVARUNETS, Rockville, MD (US); Robert A. VAN WYK, St. Pete Beach, FL (US)

(73) Assignee: ElectroMedical Associates LLC, Bethesda, MD (US)

(21) Appl. No.: 14/595,505

(22) Filed: Jan. 13, 2015

Related U.S. Application Data
(60) Provisional application No. 61/964,775, filed on Jan. 14, 2014.

(51) Int. Cl. A61B 18/14

(52) U.S. Cl. CPC A61B 18/14; A61B 2018/00071

(57) ABSTRACT

Conventional electrosurgical devices used in a conductive fluid environment have one or more electrodes at the active electrode potential, and one or more return electrodes at the return potential. The shape of the electric field and the current density for a given power setting are determined primarily by the configuration, size and relative locations of the active and return electrodes and of dielectric elements surrounding and separating the electrodes. Two potentials are supplied to the site by the electrosurgical power supply. Disclosed herein are mechanisms and methods for enhancing the effectiveness of an electrosurgical device in a conductive fluid environment that utilize an additional electrode (i.e., an auxiliary electrode) that designates a third potential between that of the active and return electrodes.

### PRIORITY

This application claims the benefit of U.S. Provisional Application No. 61/964,775 filed Jan. 14, 2014, the contents of which are hereby incorporated by reference in their entirety.

### FIELD OF THE INVENTION

The present invention relates generally to the field of electrosurgery, and more particularly to high efficiency surgical devices, systems and methods that use radio frequency (RF) electrical power for the cutting, bulk removal by vaporization (ablation), and coagulation of soft tissue in electrically conductive fluids. Such systems and instruments find particular utility in the context of minimally invasive surgery.

### BACKGROUND OF THE INVENTION

Least invasive surgical techniques have gained significant popularity due to their ability to accomplish outcomes with reduced patient pain and accelerated return of the patient to normal activities. Least invasive electrosurgical devices use RF energy for the bulk removal by vaporization (ablation), coagulation/desiccation, cutting and treatment of soft tissue in a conductive liquid environment. They are also used in other forms of soft tissue treatment such as cutting, shrinking, lesion formation, sculpting and thermal treatment in dry and semi-dry fields, as well as with conductive and non-conductive irrigants.

The effectiveness of electrosurgical devices can have a strong effect on clinical efficacy and patient safety. Accordingly, various methods have been employed to enhance effectiveness, efficiency, and efficacy. One such method for improving effectiveness is described by Carmel et al. in U.S. Pat. Nos. 7,563,261, 7,566,333 and 8,308,724, wherein the effectiveness of both monopolar and bipolar electrosurgical devices is shown to be increased through the addition of one or more auxiliary, electrically conductive elements in addition to the standard active and return electrodes, albeit one not electrically connected to a power source. As the auxiliary element has “floating-potential”, it is characterized by Carmel et al. as a “floating-potential electrode”. In the context of electrosurgery, the addition of such a floating-potential electrode increases the field intensity in the region surrounding the active electrode so as to increase the portion of the applied power that results in clinical benefit. Such devices are also effective at reduced power.

Curtis et al., in U.S. Pat. Nos. 8,518,034 and 8,394,089, describe an electrosurgical device having three electrodes, namely a single active electrode and two optional return electrodes, that are connected to an electrosurgical generator through a switching circuit such that only two of the three electrodes are directly connected to the electrical power source (activated) at any given instant of time. The switching circuit selectively directs the RF energy to either one pair chosen from the three available electrodes, or to another pair chosen from of the same three available electrodes. Using this approach, energy from the power source is directed to a first pair of electrodes when a first RF waveform is chosen, and to a second pair of electrodes when a second RF waveform is chosen. One electrode, the active, is always connected; however, either of the two return electrodes, a first in close proximity to the active electrode, or a second that is larger and further removed from the active electrode, is selected depending on the clinical effect desired. Selection of the first return (the one in close proximity) gives increased current density at the active electrode and thus is preferred when vaporizing tissue. Selection of the second gives a larger coagulation region with lower current densities and thus is preferred when coagulation and desiccation is desired. However, even though the first return electrode is electrically disconnected from the power supply during the coagulation process described by Curtis et al, it is nevertheless submerged in a conductive liquid and therefore in the return current path which encompasses the conductive liquid continuum.

Goble, in U.S. Pat. Nos. 7,491,199 and 6,966,907, describes an electrosurgical system in which the cutting and coagulation waveforms are delivered to different electrodes of the electrosurgical instrument. Goble describes an electrosurgical system having a device with three electrodes and an electrosurgical generator that provides RF energy of a first cutting waveform to a first pair of electrodes, and RF energy of a second coagulating waveform to a second pair of electrodes, with the generator allowing both the cutting and coagulating waveforms to be provided to their corresponding electrodes simultaneously. The RF generator system according to Goble includes at least first and second sources of RF power, operating at different frequencies, with the first source of RF power being adapted to deliver the first cutting waveform, and the second source of RF power being adapted to deliver the second coagulating RF waveform In the combined mode, the controller is operable to cause the generator system to deliver both the first and the second RF waveforms simultaneously. However, unlike the previously described electrosurgical devices, the Goble system requires a complex, specialized generator. Accordingly, the three-electrode device and system described by Goble cannot be implemented with the general-purpose electrosurgical generators present in virtually all modern operating rooms.

In sum, although multi-electrode approaches have yielded improvements in device effectiveness, there nevertheless remains a need in the art to further enhance the efficiency effectiveness, and efficacy of minimally invasive electrosurgical procedures. The present invention addresses this need through the multi-electrode devices and methods described herein.

### SUMMARY OF THE INVENTION

A primary objective of the present invention is to provide means and methods for improving the efficiency, effectiveness, and efficacy of multi-electrode electrosurgical devices. R is a further objective of the present invention to provide a highly efficient, minimally invasive electrosurgical device, system and method capable of overcoming the deficiencies discussed above. More particularly, in view of the ever-present need in the art for improvements in electrosurgical device and system efficiency, it is an objective of the present invention to provide a highly efficient and efficacious electrosurgical instrument and system suitable for the cutting, vaporization, coagulation and thermal modification of tissue in the presence of electrically conductive liquids such as saline. To that end, the present invention provides a method for improving the effectiveness of electrosurgical devices having three electrodes immersed in an electrically conductive fluid such as saline, bodily fluids, and the like. According to the principles of the present invention, all three electrodes are always (permanently) electrically connected to a power source using an appropriate electrical network (i.e., hard-wired). Central to the present invention is the discovery that connection in this manner, when coupled with electrical circuitry in accordance with the principles of the invention, results in the favorable modification of the distribution of energy in the conductive liquid surrounding the treatment portion of the electrosurgical device in such a way as to enhance the device performance and patient outcome.

Aspects and embodiments of the present invention in accordance with the foregoing objectives are as follows:

In one embodiment, the present invention provides an electrosurgical device having increased effectiveness wherein the device is characterized by:
a. a handle portion;
b. an elongate shaft having a proximal end connected to the handle portion and a distal end region comprising a plurality of electrically conductive members including:
   i. at least one active electrode having a proximal end and a distal end that is positioned on the elongate shaft at or near its distal end,
   ii. at least one auxiliary electrode having a proximal portion and a distal portion that is positioned on the elongate shaft in close proximity to the at least one active electrode,
   iii. at least one insulating dielectric member disposed between each of the at least one active electrodes and each of the at least one auxiliary electrodes, and
   iv. a first conductor connected to the at least one active electrode,

wherein:
the distal portion of the at least one auxiliary electrode is positioned in close proximity to one end of the at least one active electrode so as to increase the energy density in the region surrounding the active electrode;
each of the at least one active electrodes is configured for uninterrupted electrical connection to a power source, preferably an RF power source, via the first conductor; and
each of the at least one auxiliary electrodes is electrically connected to the first conductor via circuitry that decreases the power delivered to the at least one auxiliary electrode relative to the at least one active electrode.
   In a preferred embodiment the circuitry contains at least one resistor having a value between 20 Ohms and 100 mega Ohms, preferably between 300 Ohms and 5 kOhms, more preferably between 500 Ohms and 3 kOhms.

In another embodiment, the present invention provides an electrosurgical device having increased effectiveness characterized by:
a. a handle portion;
b. an elongate shaft having a proximal end connected to the handle portion and a distal end region comprising a plurality of conductive members including;
   i. at least one active electrode having a proximal end and a distal end that is positioned on the elongate shaft at or near the distal end of the shaft,
   ii. at least one auxiliary electrode having a proximal portion and a distal portion that is positioned on the elongate shaft in close proximity to the at least one active electrode,
   iii. at least one insulating dielectric member disposed between each of the at least one active electrodes and each of the at least one auxiliary electrodes,
   iv. a return electrode, preferably comprising a ring electrode mounted about the elongate shaft; and
   v. a first conductor connected to the at least one active electrode and a second conductor connected to the at least one return electrode,

wherein:
the distal portion of the at least one auxiliary electrode is positioned in close proximity to one end of the at least, one active electrode so as to increase the energy density in the region surrounding the active electrode;
each of the at least one active electrodes is configured for uninterrupted electrical connection to the power source via the first conductor; and
each of the at least one auxiliary electrodes is electrically connected to the second conductor via circuitry.
   In a preferred embodiment the circuitry contains at least one resistor having a value between 0.1 Ohm and 2 kOhms, preferably between 0.1 Ohm and 100 Ohms, more preferably between 0.1 Ohm and 20 Ohms.

In one illustrative embodiment, the electrosurgical device is a three-electrode electrosurgical device including (at least) three distinct conductive members (or “electrodes”), namely an active electrode, an auxiliary electrode, and a return electrode, all of which are permanently electrically connected to the same power source through the circuitry. In use, the electric field in proximity to the first conductive member (i.e., the “active electrode”) can be enhanced by the presence of a second conductive member (i.e., the “auxiliary electrode”) during tissue vaporization. The degree of enhancement is determined by the potential of the auxiliary electrode, which is in turn determined by the associated circuitry. The multi-electrode electrosurgical device of the present invention is designed to be immersed in an electrically conductive fluid and thus can simultaneously have multiple enhanced performance characteristics including: (a) enhanced ablation (vaporization) rate, (b) improved coagulation capabilities and (c) rapid ignition.

In contrast to the devices of the prior art, such as the above-described Carmel devices, all the electrodes of the multi-electrode device of the present invention are permanently electrically connected (i.e., hard-wired) to the power source. In addition, in contrast to the above-described Goble and Curtis devices, the multi-electrode device of the present invention (a) requires no switching circuit to selectively direct the RF energy to either one pair out of the three electrodes or to another pair out of the same three electrodes, (b) has at least three electrodes, all of which are always electrically connected (hard-wired) to a single power source and, (c) only utilizes one source of RF output power. Furthermore, in contrast to Goble, the present invention does not require a complex generator system and may in fact be adapted to operate with the general purpose generators found in virtually all modern operating rooms. In this manner, the present invention provides certain improvements in effectiveness and outcome relative to the prior art.

To that end, embodiments of the multi-electrode electrosurgical device of the present invention can be used with general-purpose electrosurgical units (ESU) as well as with dedicated ESU's. The specially designed network connecting the electrodes of the electrosurgical device in accordance with the principles of the present invention can be physically located in the device hand piece, in the electrical cord, in the electrical connector to the ESU, or incorporated in the ESU. It is accordingly yet a further objective to provide an electrosurgical system comprised of an electrosurgical device of the present invention in combination with an electrosurgical generator, more particularly an RF generator. In addition, electrosurgical devices designed in accordance with the principles of the present invention can be made in various configurations, with or without on-board aspiration and irrigation. The innovative approach of incorporating one or more auxiliary conductive elements and a network designed for favorably altering the distribution of energy may be advantageously applied to electrosurgical devices used with remotely located return electrodes (i.e., monopolar) and to devices having a return electrode located on the device itself (i.e., bipolar or multipolar).

These and other aspects of the present invention are described herein below with reference to a number of specific embodiments. However, it is to be understood that both the foregoing summary of the invention and the following detailed description are of a preferred embodiment, and not restrictive of the invention or other alternate embodiments of the invention. Further objects and features of the invention will become more fully apparent when the following detailed description is read in conjunction with the accompanying figures and examples.

### BRIEF DESCRIPTION OF THE FIGURES

Various aspects and applications of the present invention will become apparent to the skilled artisan upon consideration of the brief description of figures and the detailed description of the present invention and its preferred embodiments that follows:

FIG. 1 schematically depicts an electrosurgical device in accordance with the principles of the instant invention.

FIG. 2 is a schematic for the dedicated circuitry designed in accordance with the principles of the instant invention for use with the device of FIG. 1.

FIG. 3 is a depiction of the electrosurgical device of FIG. 1 showing the current flow paths that arise when connected to the circuitry of FIG. 2

FIG. 4A is a plan view of an electrosurgical device useful for numerical modeling.

FIG. 4B is a side elevational view of the objects of FIG. 4A.

FIG. 4C is a perspective view of the objects of FIG. 4A.

FIG. 4D is a side elevational sectional view of the objects of FIG. 4A at location A-A of FIG. 4A.

FIG. 5A is a plan view of the device of FIG. 4A with bubble and spark analysis elements added.

FIG. 5B is a side elevational view of the objects of FIG. 5A.

FIG. 5C is a perspective view of the objects of FIG. 5A

FIG. 5D is a side elevational sectional view of the objects of FIG. 5A at location A-A of FIG. 5A.

FIG. 6 is a numerical analysis plot of the current density surrounding the distal end of a conventional monopolar electrosurgical device when submerged in a conductive fluid.

FIG. 7 is a numerical analysis plot of the current density surrounding the distal end of a monopolar electrosurgical device formed in accordance with the principles of the present invention when submerged in a conductive fluid.

FIG. 8 is a numerical analysis plot of the current density surrounding the distal end of a conventional bipolar electrosurgical device when submerged in a conductive fluid.

FIG. 9 is a numerical analysis plot of the current density surrounding the distal end of a multipolar electrosurgical device formed in accordance with the principles of the present invention when submerged in a conductive fluid.

FIG. 10 schematically depicts illustrative circuitry for an alternate embodiment of the present invention.

FIG. 11 depicts the current flow of the electrosurgical device of FIG. 1 when connected to the circuitry of FIG. 10.

FIG. 12 is a numerical analysis plot of the current density surrounding the distal end of a conventional bipolar electrosurgical device when submerged in a conductive fluid at the moment prior to the initiation of fluid vaporization and ablative discharge.

FIG. 13 is a numerical analysis plot of the current density surrounding the distal end of an alternate embodiment electrosurgical device of the present invention when submerged in a conductive fluid at the moment prior to the initiation of fluid vaporization and ablative discharge.

FIG. 14 depicts an illustrative electrosurgical device that includes an auxiliary electrode constructed in accordance with the principles of this invention.

FIG. 15 is a plan view of the distal assembly of FIG. 14.

FIG. 16 is a side elevational view of the objects of FIG. 15.

FIG. 17 is an expanded side elevational view of the proximal portion of the elements of FIG. 16.

FIG. 18A is a plan view of the distal assembly of FIG. 14.

FIG. 18B is a side elevational sectional view of the objects of FIG. 18A at location A-A of FIG. 18A.

FIG. 19 is a perspective view of the objects of FIG. 14.

FIG. 20 is an expanded view of the distal portion of the objects of FIG. 19.

### DESCRIPTION OF THE PREFERRED EMBODIMENTS

Although any methods and materials similar or equivalent to those described herein can be used in the practice or testing of embodiments of the present invention, the preferred methods, devices, and materials are now described. However, before the present materials and methods are described, it is to be understood that the present invention is not limited to the particular sizes, shapes, dimensions, materials, methodologies, protocols, etc. described herein, as these may vary in accordance with routine experimentation and optimization. It is also to be understood that the terminology used in the description is for the purpose of describing the particular versions or embodiments only, and is not intended to limit the scope of the present invention which will be limited only by the appended claims. Accordingly, unless otherwise defined, all technical and scientific terms used herein have the same meaning as commonly understood by one of ordinary skill in the art to which the present invention belongs. However, in case of conflict, the present specification, including definitions below, will control.

In the context of the present invention, the following definitions apply:

The words “a”, “an” and “the” as used herein mean “at least one” unless otherwise specifically indicated. Thus, for example, reference to an “electrode” is a reference to one or more electrodes and equivalents thereof known to those skilled in the art, and so forth.

The term “proximal” as used herein refers to that end or portion which is situated closest to the user of the device, farthest away from the target surgical site. In the context of the present invention, the proximal end of the multi-electrode electrosurgical device includes the hand piece.

The term “distal” as used herein refers to that end or portion situated farthest away from the user of the device, closest to the target surgical site. In the context of the present invention, the distal end of the multi-electrode electrosurgical device includes the at least three conductive members.

The terms “lengthwise” and “axial” as used interchangeably herein to refer to the direction relating to or parallel with the longitudinal axis of a device. The term “transverse” as used herein refers to the direction lying or extending across or perpendicular to the longitudinal axis of a device.

The term “lateral” pertains to the side and, as used herein, refers to motion, movement, or materials that are situated at, proceeding from, or directed to a side of a device.

The term “medial” pertains to the middle, and as used herein, refers to motion, movement or materials that are situated in the middle, in particular situated near the median plane or the midline of the device or subset component thereof.

The term “rotational” as used herein refers to the revolutionary movement about the center point or longitudinal axis of the device.

The terms “tube” and “tubular” are interchangeably used herein to refer to a generally round, long, hollow component having at least one central opening often referred to as a “lumen”.

The present invention makes reference to a multi-electrode electrosurgical device. However, the term “device” may be used interchangeably with the terms “instrument” and “probe”. Such electrosurgical devices typically include a “structural member”, “elongate portion” or “shaft” that directly conducts energy to the respective electrodes. The structural member is typically elongate, of a linear or angled, and rounded, rod-like or tubular construction. The elongate shaft is preferably conductive and more preferably formed of metal or metallic material. In certain embodiments, the shaft may be hollow, including a lumen running therethrough that serves as a channel for the inner element or an aspiration path for removing gaseous and liquid ablation byproducts. The latter lumen flow may also serve to cool the device. However, non-lumened and non-aspirating inner element embodiments are also contemplated. The shaft that conducts power may be surrounded by and electrically isolated from a coaxially positioned an external metallic tubular element which may in certain embodiments be part of the return current path to the generator, a distal portion of the external metallic tubular element serving as a return electrode.

Electrosurgical devices contemplated by the present invention may be fabricated in a variety of sizes and shapes to optimize performance in a particular surgical procedure. For instance, instruments configured for use in small vascular spaces such as the brain may be highly miniaturized while those adapted for shoulder, knee and other large joint use may need to be larger to allow high rates of tissue removal. Likewise, electrosurgical instruments for use in arthroscopy, otolaryngology and similar fields may be produced with a rounded geometry, e.g., circular, cylindrical, elliptical and/or spherical, using turning and machining processes, while such geometries may not be suitable for other applications. Accordingly, the geometry (i.e., profile, perimeter, surface, area, etc.) may be square, rectangular, polygonal or have an irregular shape to suit a specific need.

The multi-electrode electrosurgical instruments of the present invention are characterized by the presence of multiple distinct conductive members or elements referred to herein as “electrodes”. In certain embodiments, such electrodes are ring electrodes, preferably manufactured by machining from bar stock or hypodermic tubing, or, for other more complex geometries, more preferably formed by metal injection molding. The respective electrodes may be, for instance, rings displaced axially on the elongate device shaft, and preferably include at least one single active, auxiliary, and return electrode, or multiples of either, both, or all three. The electrodes are preferably fabricated from a suitable metallic material such as, for instance, stainless steel, nickel, titanium, molybdenum, tungsten, and the like as well as combinations thereof. However, electrically conductive non-metals are also contemplated.

In the context of the present invention, the “active electrode” is generally disposed at the distal end of the instrument. In the context of the present invention, the respective electrodes are all connected, for example via wiring disposed within the control/handle portion of the instrument, to a power supply, for example, an externally located electrosurgical generator.

In certain embodiments, the present invention makes reference to one or more “insulators” separating the respective electrodes. As used herein, the term “insulator” refers to a electrically non-conductive element formed from a suitable dielectric material, examples of which include, but are not limited to, alumina, zirconia, and high-temperature polymers formed as solid, or non solid, such as fibers. Alternatively, the insulator may take the form of a coating utilized to cover portions of the electrode and leave others exposed. Suitable coatings may be from suitable polymeric materials applied, for instance, as a powder coat or liquid that is subsequently cured, or as a molded or extruded tube which is shrunk by heat after application. Components of multi-electrode assembly may optionally be held in place by such coatings, although a suitable adhesive cement may also be used.

In particularly preferred embodiments, the multi-electrode electrosurgical device of the present invention includes at least three distinct electrodes, namely at least one “active” electrode, “auxiliary” electrode, and “return” electrode. The prior art conventionally refers to electrosurgical devices that utilize an onboard return electrode as “bipolar” and those that utilize a separate, remotely located return electrode (often referred to as a “dispersive electrode” or “return pad”) as “monopolar”. While the present invention contemplates both configurations, such terms are perhaps inaccurate in the context of the present invention since the auxiliary electrode of the present invention has a potential that is greater than that of return electrode such that when return electrode is remotely located there are still two electrodes with different potentials mounted on the device, a characteristic of a bipolar device. Similarly, when the return electrode is mounted on the device in proximity to electrodes, the device has three electrodes each at their own potential at the device distal end making it no longer bipolar but rather tripolar or, more generally, multi-polar.

Like the overall electrosurgical instrument, the size, shape and orientation of the respective electrodes and the various active surfaces defined thereby may routinely vary in accordance with the need in the art. It will be understood that certain geometries may be better suited to certain utilities. Accordingly, those skilled in the art may routinely select one shape over another in order to optimize performance for specific surgical procedures. For example, in some embodiments, the multi-electrode electrosurgical device may have a radial symmetry with the auxiliary electrode forming the outermost radial surface at the device tip. The auxiliary electrode may completely or only partially surround the tip, and may have features to locally increase the current density such as, for instance, notches or protuberances. In other embodiments, the device tip may have a non-radial symmetry with the auxiliary electrode completely or partially surrounding the active electrode, while in other embodiments the auxiliary and active electrode form an array of protuberances with the auxiliary electrodes being interspersed in the array of active electrodes. In yet other embodiments, the active and auxiliary electrodes form an assembly having a blade-like structure useful for cutting tissue.

The active, auxiliary, and return electrodes may be formed and arranged in a variety of configurations to accomplish tissue vaporization for a range of applications and conditions. These include, but are not limited to, bulk tissue vaporization, tissue cutting, and producing holes in tissue. Because the present invention permits the field to be intensified, the time required to form steam bubbles and achieve arcing within the bubbles (i.e. ignition) is shortened.

In certain embodiments, the present invention makes reference to “conductive fluid(s)”, particularly in connection with the “wet environment” embodiments. As used herein, the term “fluid” encompasses liquids, gases and combinations thereof, either electrically conductive or non-conductive, intrinsic to the tissue or externally supplied. In the context of the present invention, the term “fluid” extends to externally supplied liquids such as saline as well as bodily fluids, examples of which include, but not limited to, blood, plasma, saliva, peritoneal fluid, lymph fluid, pleural fluid, gastric fluid, bile, and urine.

The present invention makes reference to the ablation, coagulation, vaporization and cauterization of tissue. As used herein, the term “tissue” refers to biological tissues, generally defined as a collection of interconnected cells that perform a similar function within an organism. Four basic types of tissue are found in the bodies of all animals, including the human body and lower multicellular organisms such as insects, including epithelium, connective tissue, muscle tissue, and nervous tissue. These tissues make up all the organs, structures and other body contents. The present invention is not limited in terms of the tissue to be treated but rather has broad application, including the resection and/or vaporization any target tissue with particular applicability to the ablation, vaporization, destruction and removal of tissue in joints of the body as well as musculoskeletal applications.

The instant invention has both human medical and veterinary applications. Accordingly, the terms “subject” and “patient” are used interchangeably herein to refer to the person or animal being treated or examined. Exemplary animals include house pets, farm animals, and zoo animals. In a preferred embodiment, the subject is a mammal.

Hereinafter, the present invention is described in more detail by reference to the Figures and Examples. However, the following materials, methods, figures, and examples only illustrate aspects of the invention and are in no way intended to limit the scope of the present invention. For example, while the present invention makes specific reference to electrosurgical procedures conducted in the presence of an externally applied electrically conductive fluid, it is readily apparent that the teachings of the present invention may be applied to other minimally invasive procedures. As such, methods and materials similar or equivalent to those described herein can be used in the practice or testing of the present invention.

### Utilities of the Present Invention

As discussed in greater detail below, the present invention utilizes an auxiliary electrode to intensity the electric current in select regions to thereby enhance vaporization and/or coagulation performance as desired. In certain embodiments, the auxiliary electrode allows for the creation of an additional large region of high current density adjacent to the distal treatment portion of the device so as to afford enhanced coagulation when ablating (vaporizing) tissue. Likewise, when used in coagulation mode, the presence of the high current density region created by an auxiliary electrode can enhance the effectiveness of device by creating an additional region of tissue desiccation at an electric field that will not create undesirable arcing. Embodiments of the present invention can also provide more rapid heating of the liquid and formation of bubbles and the resulting subsequent ablative discharge. As such, the time delay between the activation of the device and effective ablative discharge (ignition) may be reduced and the tissue vaporization rate may be increased due to the presence of the auxiliary electrode and its associated circuitry. As discussed in detail below, this may bring beneficial clinical effect to the patient through enhanced performance of the electrosurgical device according to the principles of this invention.

It should be noted that the present invention is not restricted to one particular field of surgery but rather finds utility in connection with a wide variety of applications, from oncological to reconstructive, cosmetic, arthroscopic, ENT, urological, gynecological, and/or laparoscopic procedures, as well as in the context of general open surgery.

As noted above, the electrosurgical instruments designed in accordance with the principles of the present invention find utility in connection with a variety of medical, both human and veterinary, applications for cutting, cauterization, coagulation, evaporation, sculpting, shrinking, smoothing, lesion formation, among others, in various types of tissue. The instruments can be used in a variety of medical procedures, like minimally invasive or open surgery, cosmetic, dental or dermatological, on the surface or inside the body. To that end, the active area of the instrument (i.e., the active element at the distal end) can take many shapes and forms, and can be configured to meet the needs of the specific procedure in such fields. Thus, for the most part, choices in geometry constitute a design preference.

Electrosurgical instruments formed in accordance with the principles of this invention generally include of a proximal handle portion and an elongate distal portion designed to be inserted into the environment of interest. The proximal end of the instrument is typically connected to an electrosurgical generator, wherein the handle portion is provided with one or more buttons (or switches or other activating elements) on the surface that control the output of the electrosurgical generator. Alternatively, the electrosurgical generator may be controlled by a foot-activated control. In either case, depending on the environment, the desires of the surgeon, and the condition being treated, instruments of the present invention can be operated continuously or intermittently, at variable powers, frequencies and intensities.

While some embodiments of the present invention are designed to operate in dry or semi-dry environments, other bipolar embodiments utilize the endogenous fluid and/or an exogenous irrigant of a “wet field” environment to transmit current to the return electrode and therethrough to the RF energy source. In certain embodiments, the “irrigant” (whether native or externally applied) is heated to the boiling point, whereby thermal tissue treatment arises through direct contact with either the boiling liquid itself or steam associated therewith. This thermal treatment may include desiccation to stop bleeding (haemostasis), and/or shrinking, denaturing, or enclosing of tissues for the purpose of volumetric reduction (as in the soft palate to reduce snoring) or to prevent aberrant growth of tissue, for instance, malignant tumors.

Liquids (either electrically conductive or non-conductive) and gaseous irrigants, either singly or in combination may also be advantageously applied to instruments for incremental vaporization of tissue. Normal saline solution may be used. Alternatively, the use of low-conductivity irrigants such as water or gaseous irrigants or a combination of the two allows increased control of the electrosurgical environment.

The electrosurgical instruments of the present invention may be used in conjunction with existing diagnostic and imaging technologies, for example imaging systems including, but not limited to, MRI, CT, PET, x-ray, fluoroscopic, thermographic, photo-acoustic, ultrasonic and gamma camera and ultrasound systems. Such imaging technology may be used to monitor the introduction and operation of the instruments of the present invention. For example, existing imaging systems may be used to determine location of target tissue, to confirm accuracy of instrument positioning, to assess the degree of tissue vaporization (e.g., sufficiency of tissue removal), to determine if subsequent procedures are required (e.g., thermal treatment such as coagulation and/or cauterization of tissue adjacent to the target tissue and/or surgical site), and to assist in the atraumatic removal of the instrument.

### Illustrative Embodiments of the Present Invention

Hereinafter, the present invention is described in more detail by reference to the exemplary embodiments. However, the following examples only illustrate aspects of the invention and in no way are intended to limit the scope of the present invention. As such, embodiments similar or equivalent to those described herein can be used in the practice or testing of the present invention.

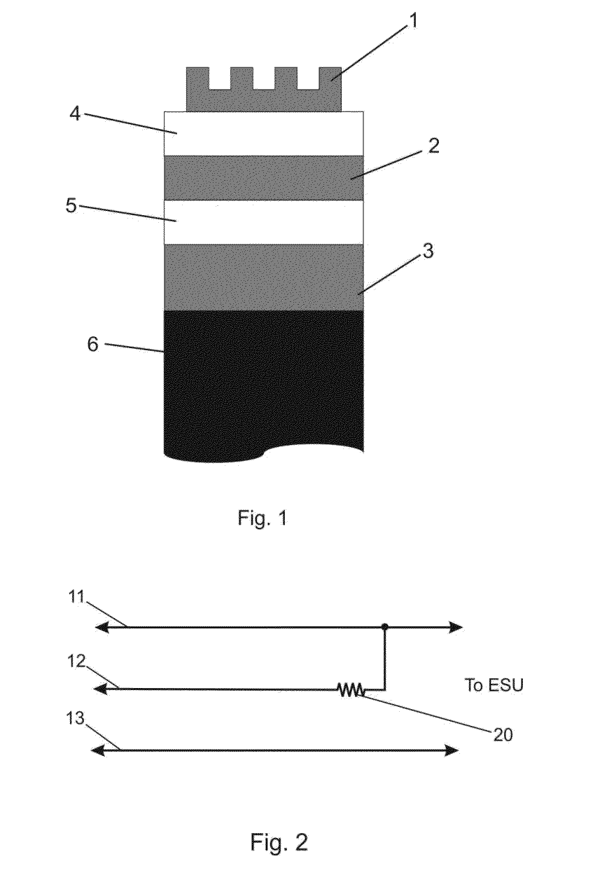

The principles of the current invention are illustrated schematically in FIG. 1 depicting the distal end of an electrosurgical device having three conductive members (electrodes) 1, 2, and 3. The conductive members 1, 2 and 3 are separated by dielectric insulators 4, 5 and 6, respectively. For illustration purposes, conductive member 1 may be referred to as the active electrode, conductive member 3 may be referred to as the return electrode, and conductive member 2 may be referred to as the auxiliary electrode.

According to the principles of the current invention, the conductive members 1, 2 and 3 are electrically connected to the power source via the circuitry shown schematically in FIG. 2, wherein conductor 11 is connected to conductive member 1 (the active electrode), conductor 12 is connected to conductive member 2 (the auxiliary electrode), and conductor 13 is connected to conductive member 3 (the return electrode). Conductor 12 is connected to conductor 11 through resistor 20. In a preferred embodiment, the second conductive member 2 has a potential that is less than that of first conductive member 1 (the active electrode) but greater than that of third conductive member 3 (the return electrode). However, the relative potentials are determined, among other things, by the value of resistor 20. By choosing the proper resistor value, the performance of the electrosurgical device can be beneficially enhanced. In addition, it should be noted that although the return electrode, conductive element 3, is shown in FIG. 1 as being in close proximity to the active electrode, conductive element 1, it may be optionally positioned elsewhere on the device or, alternatively, may be remotely located, for example on the patient's skin in the form of a return pad. Performance enhancement through shaping of the energy field in accordance with the principles of this invention occurs regardless of the position of return electrode 3.

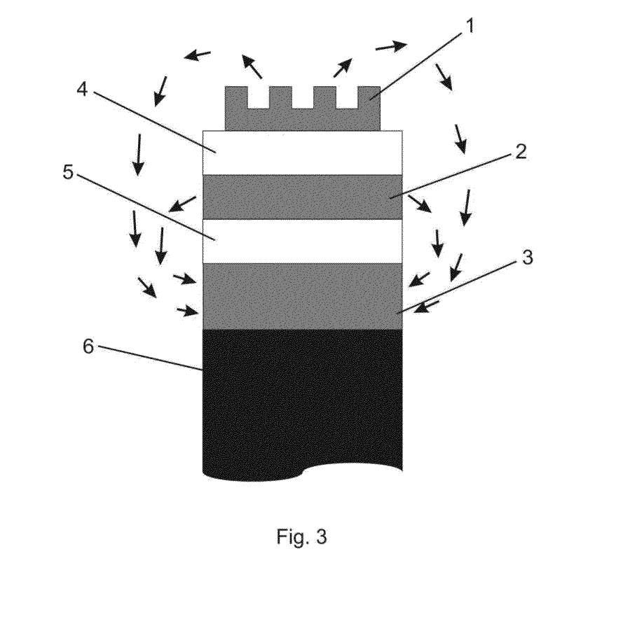

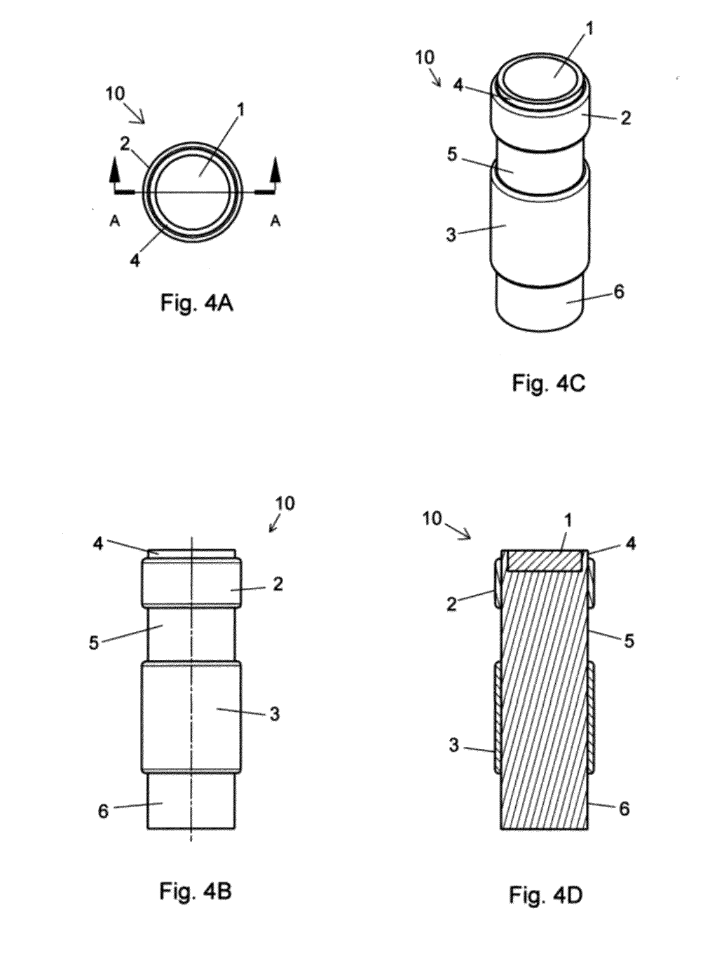

FIG. 3 depicts the current flow (indicated by arrows) for the electrosurgical device of FIG. 1 connected to an electrosurgery power supply by the circuitry of FIG. 2 when submerged in a conductive liquid. Because the potential of auxiliary electrode 2 is greater than that of return electrode 3, current flows from both electrode 1 and electrode 2 to electrode 3. The effect of the auxiliary electrode 2 on the energy distribution can be observed through numerical modeling of the distribution of current density. To that end, FIGS. 4A through 4D depict an electrosurgical device 10 used to generate current distribution figures by numerical modeling. Active electrode 1 has a distal surface flush with the distal-facing surface of insulator portion 4. Second (auxiliary) electrode 2 is in close proximity to electrode 1 separated by insulator portion 4. Third (return) electrode 3 has a larger area than electrode 2 from which it is separated by insulator portion 5, the separation distance between electrodes 2 and 3 being greater than that separating electrodes 1 and 2. As depicted, because device 10 has return electrode 3 on device 10 in close proximity to active electrode 1, device 10 is configured as what is commonly referred to as a “bipolar” device. Removing electrode 3 from device 10 and relocating it to a remote location configures device 10 into what is commonly referred to as a “monopolar” device. However, in the context of the present invention, these terms are somewhat inaccurate since auxiliary electrode 2 has a potential that is greater than that of return electrode 3 so that when return electrode 3 is remotely located there are still two electrodes with different potentials mounted on the device, a characteristic of a bipolar device. Similarly, when return electrode 3 is mounted on the device in proximity to electrodes 1 and 2, the device has three electrodes each at their own potential at the device distal end making it no longer bipolar but rather tripolar or, more generally, multi-polar.

In the following numerical analysis, the effect of auxiliary electrode 2 on the current distribution is demonstrated for device 10 with return electrode 3 remotely located and with electrode 3 in proximity on device 10.

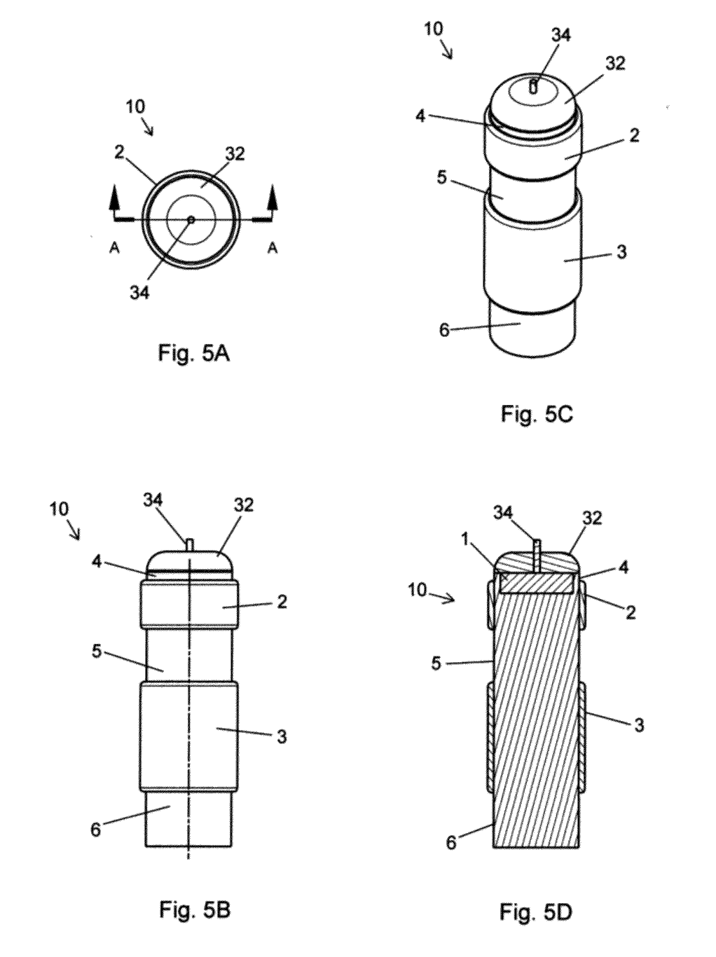

FIGS. 5A through 5D depict device 10 as modeled in the following numerical analyses. Bubble 32 is an insulating region that covers the distal portion of active electrode 1 and the distal-most surface of insulator 4. Element 34 represents a conducting channel of electrical discharge (arc or spark) passing through the insulating bubble 32 to the surrounding conductive liquid-tissue.

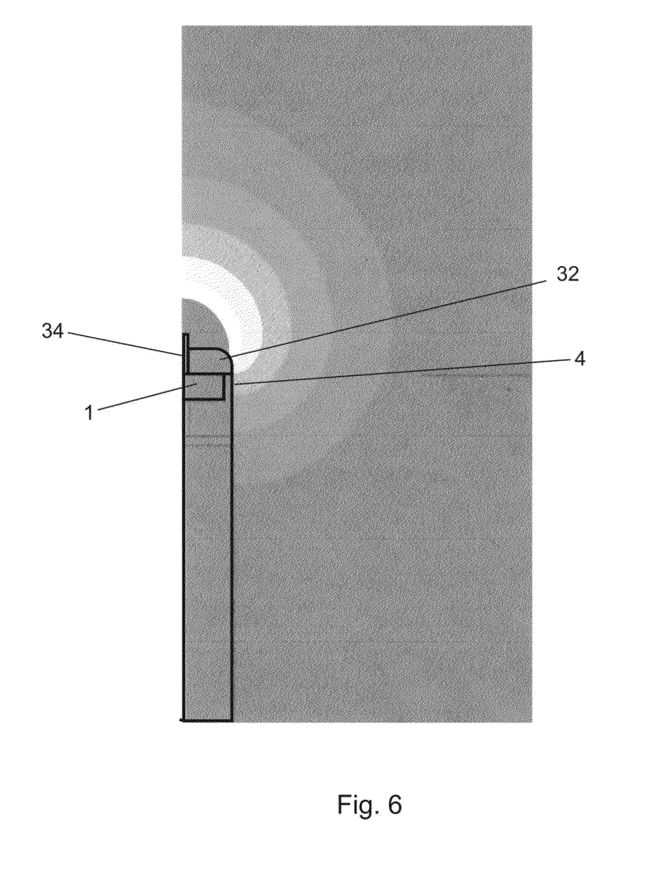

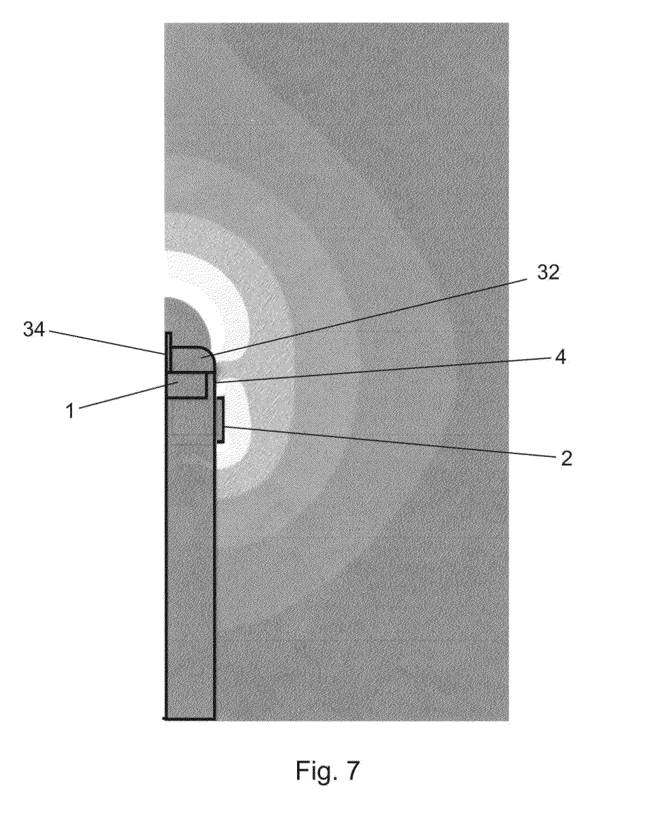

FIG. 6 depicts a numerical analysis plot of the current density surrounding the distal end of a device 10 fabricated without auxiliary electrode 2 and with return electrode 3 remotely located, i.e., the configuration of a standard monopolar device, when disposed in a conductive liquid. FIG. 6 represents one half of a section view through the device modeled as depicted in FIG. 5D. The current density distribution is indicated by shading of the conductive fluid portion surrounding device 10, wherein lightly shaded areas correspond to areas at a higher current density than more darkly shaded regions, with the exception of the dark area adjacent to bubble 32 and arc 34 where the current density is highest, as expected. FIG. 7 depicts a numerical analysis plot of the current density surrounding the distal portion of a device 10 configured to include auxiliary electrode 2, when disposed in a conductive liquid, wherein electrodes 1 and 2 and remotely located electrode 3 are all connected to a common electrosurgical power supply as depicted in FIG. 2 and current flow is as depicted in FIG. 3. The addition of auxiliary electrode 2 creates a second region of high current density (light area). Although the shading of the region adjacent to bubble 32 and arc 34 and the region surrounding auxiliary electrode 2 have the same shading, the current density in proximity to auxiliary electrode 2 is less than that of the other distal region, the degree of the difference being determined by resistor 20 of the circuitry of FIG. 2, among other factors. The additional large region of high current density adjacent to the distal treatment portion of device 10 may give enhanced coagulation when ablating (vaporizing) tissue. When used in coagulation mode, the presence of the high current density region created by auxiliary electrode 2 will enhance the effectiveness of device 10 by creating an additional region of tissue desiccation at an electric field that will not create undesirable arcing.

The effect of auxiliary electrode 2 is heavily, though not solely, determined by the value of resistor 20. Very high values of resistor 20 will limit current flow from electrode 2 to the electrosurgical power supply. In such cases, the potential of electrode 2 will be determined primarily by its position in the conductive liquid; in other words, it will have a virtually floating potential and act in accordance with the principles of the previously described Carmel devices (see U.S. Pat. Nos. 7,563,261, 7,566,333 and 8,308,724, the contents of which are incorporated by reference herein). Beneficial effects will arise due to the circuitry of the present invention and through the previously cited beneficial effects of a “floating potential” electrode. As the value of resistor 20 is decreased, this floating electrode effect is also decreased as current flow from electrode 2 increases. At very low values for resistor 20, electrode 2 begins to function as an additional active electrode. In this case there will be two active electrodes 2 and 3 and accordingly an enhanced ablation capability. The desired modification by electrode 2 of the current distribution in the region in proximity to active electrode 1 may be, therefore, achieved through positioning of auxiliary electrode 2 and the value of resistor 20. In order to achieve the coagulation enhancement previously herein described, the value for resistor 20 should range is between 20 Ohm and 100 mega Ohms, more preferably between 300 Ohm and 5 kOhm, and even more preferably between 500 Ohm and 3 kOhm.

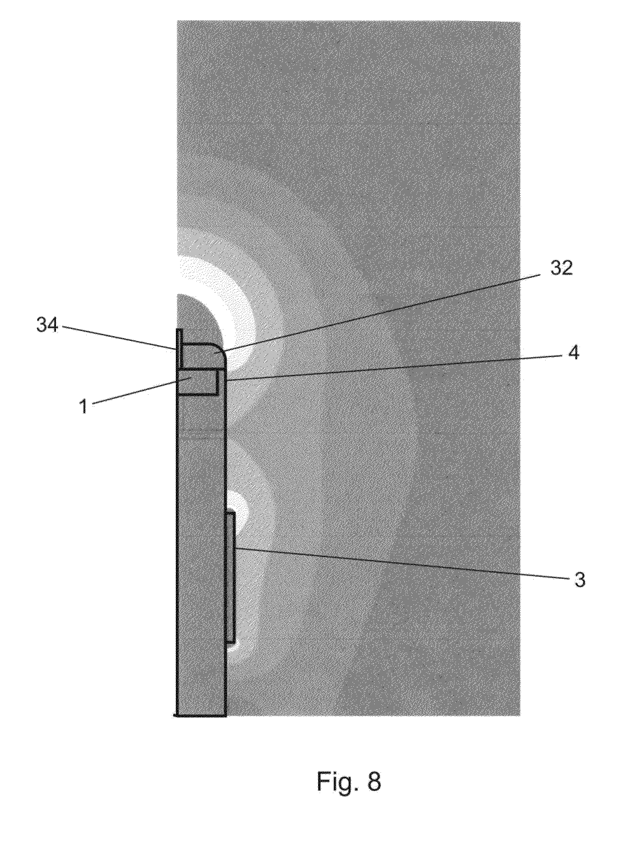

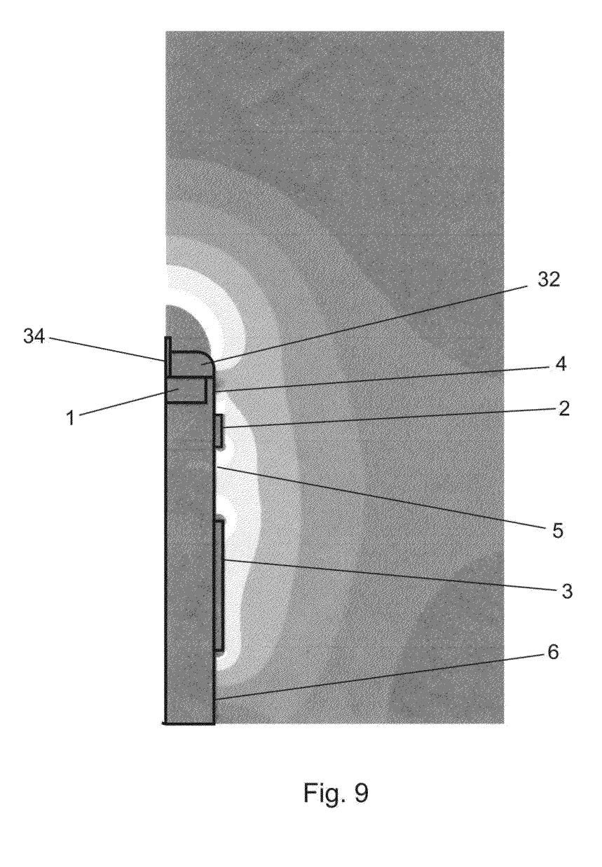

FIGS. 8 and 9 demonstrate the effect of adding auxiliary electrode 2 to a device having return electrode 3 in proximity on device 10 (FIGS. 4 and 5). FIG. 8 depicts a numerical analysis plot of the current density surrounding the distal end of a device 10 having return electrode 3 mounted on the device in proximity to active electrode 1, i.e., a conventional bipolar device. The current distribution for this configuration is similar to that of the monopolar device 10 shown in FIG. 6 with a little intensification in the distal region due to the presence of return electrode 3. FIG. 9 depicts the current distribution when auxiliary electrode 2 is added to the device 10 of FIG. 8. A second region of high intensity is created surrounding electrode 2, electrode 3 and in the region between electrodes 2 and 3. As with device 10 as depicted in FIG. 7, coagulation during ablation (vaporization) of tissue would be enhanced. More importantly, the effectiveness of the device when used in coagulation mode would be enhanced by the presence of this second region of high current density at a lower potential than that of active electrode 1. The current density in the region surrounding electrode 2, electrode 3 and the region between will be determined by the value of resistor 20 (FIG. 2) along with other factors including the distance between electrodes 2 and 3.

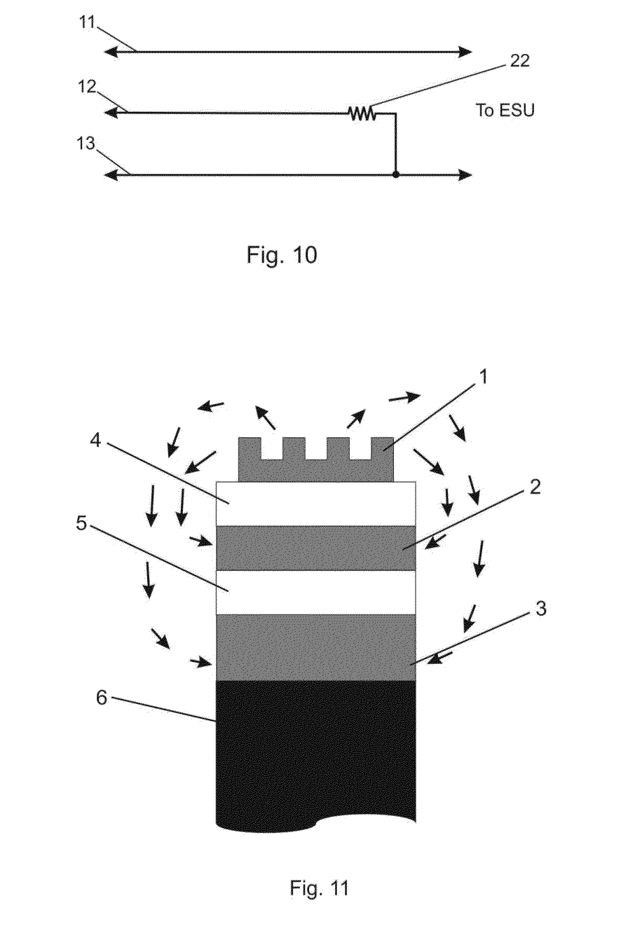

While the beneficial effects of auxiliary electrode 2 in the embodiment previously herein described have been through enhanced coagulation, in other embodiments the vaporization performance may be enhanced. Circuitry for vaporization enhancement is depicted in FIG. 10 wherein conductor 12 is connected to conductor 13 (connected to return electrode 3) through resistor 22. Referring to FIG. 11, in which current flow is indicated by arrows, current flow now is from active electrode 1 to auxiliary electrode 2 and return electrode 3. The relative portion of the return current flowing to electrodes 2 and 3 is determined by external factors including the value of resistor 22, and the relative proximity of electrode 2 to electrode 1 and electrode 3.

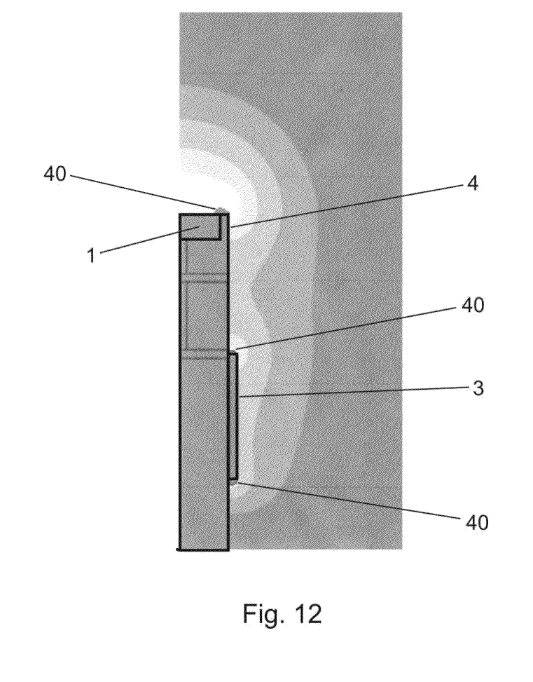

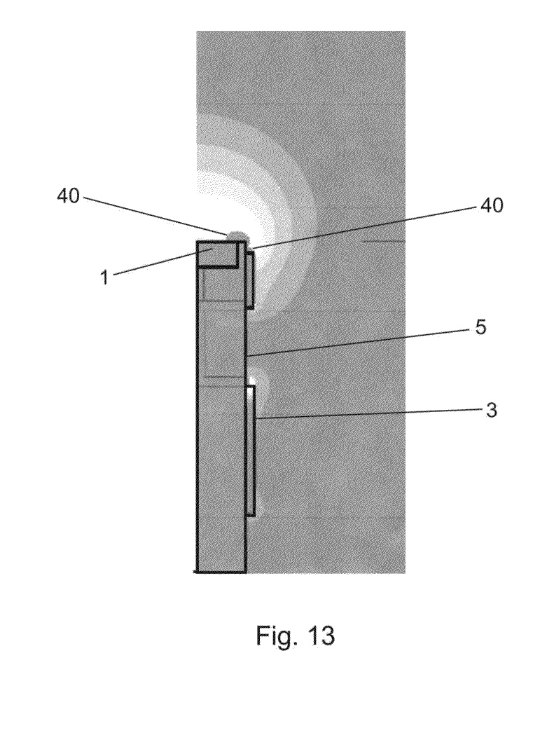

FIG. 12 depicts the current density surrounding a conventional bipolar device 10, including a return electrode 3 positioned in close proximity to active electrode 1, at the instant prior to formation of bubbles at active electrode 1 and the subsequent associated ablative discharge. The darkly shaded region of highest current density 40 is found at the periphery of electrode 1 where it abuts insulator 4, and at the proximal and distal ends of return electrode 3. The current density is indicated by shading as above, with regions of lighter shading having higher field intensity than those with darker shading. FIG. 13 depicts the current density distribution in the conductive fluid surrounding device 10 of FIG. 12 when an auxiliary electrode 2 is added and connected to an electrosurgical generator through the circuitry of FIG. 10 resulting in current flow as depicted in FIG. 11. As compared to FIG. 12, a much larger region of highest current density is created at the periphery of active electrode 1 where it abuts insulator 4, and an additional region of highest current density is formed at the distal end of auxiliary electrode 2. The intensification of the electric current in these regions due to the presence of auxiliary electrode 2 with its associated circuitry results in more rapid heating of the liquid and formation of bubbles and the resulting subsequent ablative discharge. The time delay between the activation of the device and effective ablative discharge (ignition) is reduced and the tissue vaporization rate is increased because of the presence of auxiliary electrode 2 and its associated circuitry depicted in FIG. 10. This may bring beneficial clinical effect to the patient through enhanced performance of the electrosurgical device according to the principles of this invention.

As with the previously described embodiment, the effect of auxiliary electrode 2 will again be heavily, though not solely, determined by the value of resistor 22. Very high values of resistor 22 will again limit current flow from electrode 2 to the electrosurgical power supply. In such cases, the potential of electrode 2 is again determined primarily by its position in the conductive liquid; in other words, it will have a virtually floating potential and act in accordance with the principles of the previously described Carmel devices (see U.S. Pat. Nos. 7,563,261, 7,566,333 and 8,308,724, the contents of which are incorporated by reference herein). Beneficial effects will again arise due to the circuitry of the present invention and through the previously cited beneficial effects of a floating potential electrode. Moreover, as noted above, at very low values for resistor 22, electrode 2 begins to function as an additional return electrode. As the potential of auxiliary electrode 2 is decreased to near that of return electrode 3, the minimum distance between active electrode 1 and auxiliary electrode 2 must be increased since arcing between the electrodes is undesirable. Accordingly, in this instance, it is desirable to select values for resistor 22 that produce intensification of the electric field in close proximity to active electrode 1 without arcing between the electrodes. The value of resistor 22 required to achieve this effect will depend on other characteristics of device 10 and the electrosurgical generator with which it is used. When auxiliary electrode 2 is connected through resistor 22 to conductor 13 and therethrough to return electrode 3, a preferred range of values for resistor 22 is between 0.1 Ohm and 2 kOhm, more preferably between 0.1 Ohm and 100 Ohm, and still more preferably between 0.1 Ohm and 20 Ohm. In other embodiments low resistance values may be provided by the electrical properties of conductor 12 itself without an additional discrete resistive component.

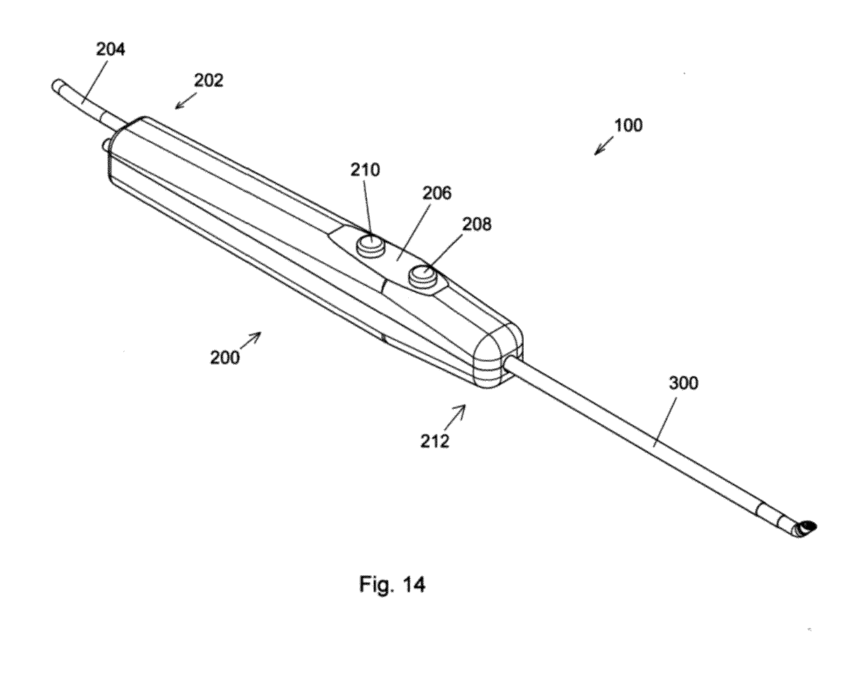

Referring to FIG. 14, which depicts an electrosurgical device 100 with an auxiliary electrode formed in accordance with the principles of this invention, device 100 has a proximal portion forming a handle 200 having a proximal end 202 from which passes cable 204 which connects to an electrosurgical generator (not shown), a top surface 206 having a first button 208 and a second button 210, and a distal end 212 from which protrudes distal assembly 300.

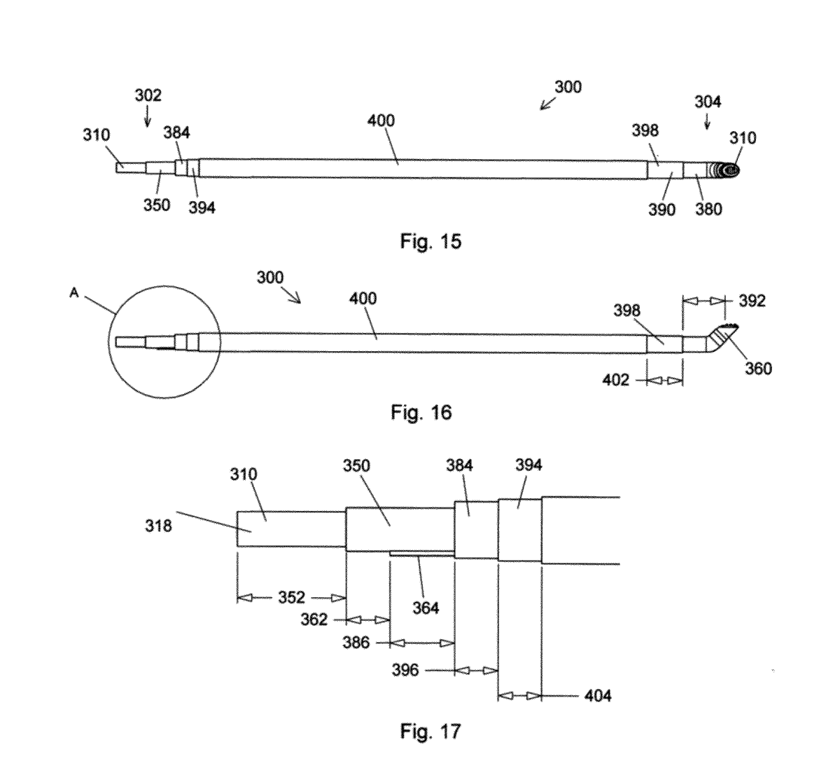

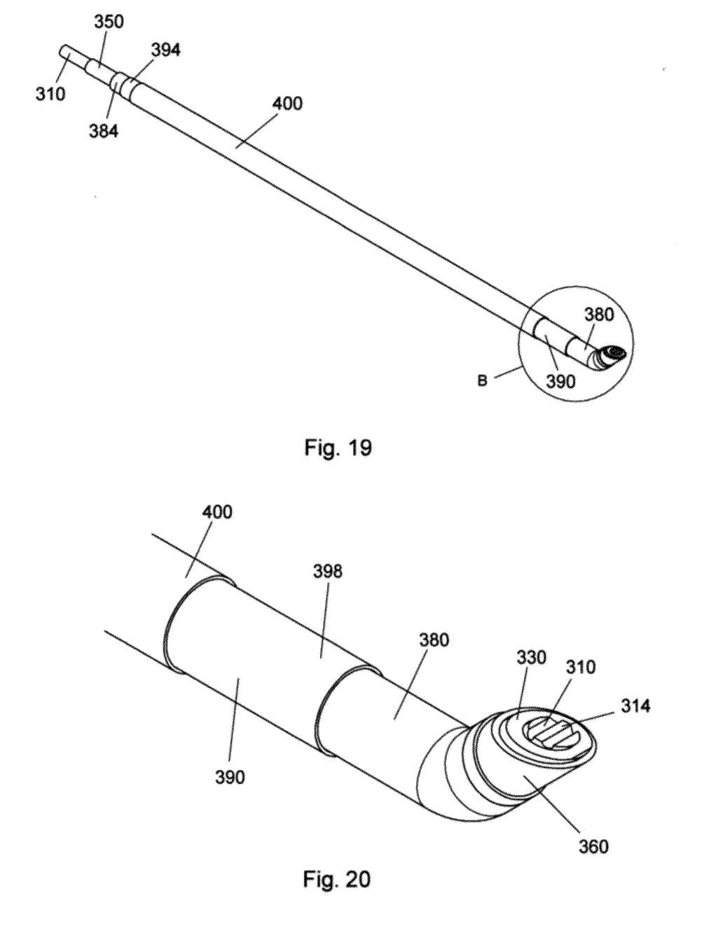

Referring now to FIGS. 15 through 19, distal assembly 300 has a proximal end 302 and a distal end 304. Proximal end 302 is mounted to handle 200 and electrically connected via means within handle 200 and cable 204 to an electrosurgical generator (not shown). Distal assembly 300 is formed of coaxially positioned conductive and dielectric members, the dielectric members electrically isolating the electrically conductive members, and the electrically conductive members forming a current path between circuitry within handle 200 and electrode elements at distal end 304 of member 300. Active electrode member 310 has a cylindrical distal portion 312 with a distal-most surface 314 forming an ablating surface, and a proximal end 318 which is connected by means within handle 200 to the electrosurgical generator. Cylindrical distal portion 312 of active electrode member 310 is surrounded by an insulator 330 that has a distal portion 332 terminating in distal-most surface 334, and a proximal portion 336 separated from distal portion 332 by flange portion 338. Dielectric member 350 at its distal end overlaps proximal portion 336 of insulator 330 and extends proximally to terminate distance 352 from the proximal end 318 of active electrode member 310. Insulator 330 and dielectric member 350 together insulate active electrode member 310 except for proximal end 318 and distal-most surface 314 and its immediately adjacent region. Auxiliary electrode 360 is mounted to insulator 330 and surrounds distal portion 332 of insulator 330. Conductive member 370 is electrically connected to auxiliary electrode 360 at its distal end 372 and extends to a proximal end 364 terminating distance 362 from the proximal end of dielectric member 350. Auxiliary electrode 360 is electrically connected via conductive member 370 to circuitry within handle 200. Dielectric member 380 has a distal end 382 which overlaps flange 338 of insulator 330 and a small portion of the proximal-most portion of auxiliary electrode 360. Dielectric member 380 extends proximally to terminate at its proximal end 384 distance 386 from the proximal end of conductive member 370. Tubular conductive member 390 has a distal end that terminates distance 392 from ablating surface 314 of active electrode member 300, and a proximal end 394 that terminates distance 396 from the proximal end 384 of dielectric member 380 Tubular member 390 is connected via circuitry and other means within handle 200 and cable 204 to the electrosurgical generator. Dielectric member 400 extends at its distal end to distance 402 from the distal end of tubular member 390 so as to create uninsulated portion 398 of member 390, and at its proximal to distance 404 from proximal end 394 of conductive tubular member 390.

Ablating surface 314 of active electrode member 310 is analogous to the first conductive element 1 of FIG. 1; auxiliary electrode 360 is analogous to the second conductive element 2 of FIG. 1; and the uninsulated portion 398 of conductive tubular element 390 which functions as a return electrode is analogous to the third conductive element 3 of FIG. 1. Their respective conductive paths are analogous to conductive elements 11, 12 and 13 respectively and connect with circuitry of FIG. 2 or 10.

In use, depressing first button 208 causes RF energy having a first predetermined power level and waveform to be supplied to ablating surface 314 of active electrode 310; depressing second button 210 causes RF energy having a second predetermined power level and waveform to be supplied to ablating surface 314 of active electrode 310.

RF current supplied by the electrosurgical generator to ablating surface 314 of active electrode 310 returns to the generator by conductive tubular element 390 via circuitry within handle 200 and cable 204, the uninsulated portion 398 of element 390 serving as a return electrode in contact with the tissue and conductive fluid at the site. Auxiliary electrode 360 is connected via conductive element 370, circuitry within handle 200 and cable 204 to the electrosurgical generator, the conductive path containing circuitry previously herein described and shown in FIGS. 2 and 10.

In the preferred embodiment depicted, device 100 has a return electrode 398 located on the device. In this configuration, the circuitry of FIG. 2 or FIG. 10 may be located within handle 200 of device 100 since both active and return circuits are present. In another preferred embodiment, device 100 is used with a remotely located return electrode (return pad) such that the return electrode 398 and its associated conduction path is thereby eliminated. If auxiliary electrode 360 is connected to circuitry as in FIG. 2, the connective circuitry may be located within handle 200. If auxiliary electrode 360 is connected to the remotely located return electrode conductive path as shown in FIG. 10, the circuitry may be located in the electrosurgical generator or, alternatively, may be housed in an adapter located outside the generator.

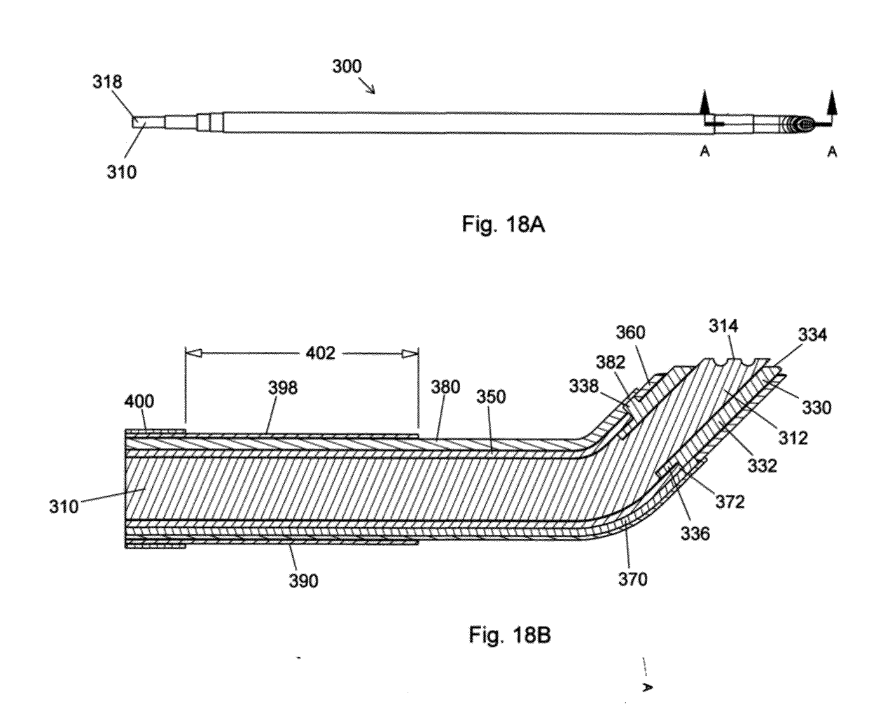

In another embodiment in which auxiliary electrode 360 is connected to active electrode 310 by a resistive element according to the circuitry of FIG. 2, the resistive element may be in the form of a resistive element configured like resistor 330 depicted in FIG. 18 but made from a conductive ceramic. The resistivity of certain ceramic materials (frequently called “lossy” ceramics”) can be modified to have finite electrical resistivity rather than being a near perfect insulator. In a preferred embodiment, the dielectric material of insulator 330 is replaced by a ceramic material having a predetermined resistivity so that auxiliary electrode 360 is electrically connected to active electrode 310 through what in this embodiment is resistive element 330, thereby eliminating the need for the external circuitry according to FIG. 2. Alternatively, the dielectric may be a suitable polymeric material. In another embodiment in which resistive element 330 is used, auxiliary electrode 360 is eliminated, the portion of the external surface of resistive element 330 in contact with the conductive fluid acting as the auxiliary electrode.

While the embodiments herein described use purely resistive elements in the connection circuitry for the auxiliary electrode other embodiments are anticipated in which other types of components or networks including capacitors, inductors, switches, tuned circuits, diodes resistors and transformers either singly or in combination are used in the connection circuit, such embodiments being within the scope of this invention. Indeed, any electrosurgical device having an electrode located in proximity to the active electrode and that electrode having a potential between that of the active electrode and the return electrode is within the scope of the current invention provided the electrode is electrically connected through circuitry to the active or return electrode by means of passive or active electrical networks, either lumped or distributed.

When used in a fluid filled environment a conductive irrigant such as standard saline or a nonconductive irrigant like sterile water or glycine may be used. When nonconductive irrigants are used, contamination of the fluid present at the site by blood and other bodily fluids makes the fluid sufficiently conductive for auxiliary electrode 360 to have a beneficial effect.

While the principles of the instant invention have been described for a device submerged in a conductive fluid environment, devices and systems constructed in accordance with the principles of this invention may be advantageously used in dry and semi-dry environments using bodily fluids or externally supplied irrigant. In a preferred embodiment, device 100 is equipped with a conduit connected to an external irrigant supply such that irrigant is supplied to the region adjacent to active electrode 314 and auxiliary electrode 360. In another preferred embodiment, device 100 is equipped with both an irrigant supply conduit and also an aspiration channel connected to an external vacuum source so as to allow device 100 to remove bubbles and debris from the treatment site.

### INDUSTRIAL APPLICABILITY

As noted previously, the present invention is directed to a multi-electrode electrosurgical device including at least three distinct conductive members (or “electrodes”), namely an active electrode, an auxiliary electrode, and a return electrode, all of which are permanently electrically connected to the power source through the circuitry (i.e., hard-wired), that yields multiple enhanced performance characteristics including: (a) enhanced ablation (vaporization) rate, (b) improved coagulation capabilities and (c) rapid ignition. Although described in detail with respect to procedures that take place in the presence of an externally supplied electrically conductive fluid, such as saline, it will be readily apparent to the skilled artisan that the utility of the present invention extends to other minimally invasive endoscopic interventions.

Conventional electrosurgical devices used in a conductive fluid environment have one or more electrodes at active electrode potential, and one or more return electrodes at the return potential. The shape of the electric field and the current density for a given power setting are determined primarily by the configuration, size and relative locations of the active and return electrodes and of dielectric elements surrounding and separating the electrodes. Two potentials are supplied to the site by the electrosurgical power supply. The present invention increases the effectiveness of electrosurgical devices in a conductive fluid environment by adding an electrode (auxiliary electrode) that is at a third potential between that of the active and return electrodes. The return electrode may be in proximity on the device, or remotely located as with a return pad. The auxiliary electrode is connected to either the active or return circuit through a resistor, the connection being made within the device, in the cabling or connector, or in the electrosurgical power supply/generator.

Critically to its success, the system of the instant invention is not complex, easily implemented, and may be used with either a standard general-purpose electrosurgical generator, or with a dedicated generator. Moreover, suitable design of the circuitry described herein allows optimization of the device performance for certain specific tasks. These may include, for instance, enhanced bulk tissue vaporization rates, the ability to operate at lower power levels than similar conventional electrosurgical devices, and/or improved coagulation during tissue vaporization.

The disclosure of each publication, patent or patent application mentioned in this specification is specifically incorporated by reference herein in its entirety. However, nothing herein is to be construed as an admission that the invention is not entitled to antedate such disclosure by virtue of prior invention.

The invention has been illustrated by reference to specific examples and preferred embodiments. However, it should be understood that the invention is intended not to be limited by the foregoing description, but to be defined by the appended claims and their equivalents.

What is claimed:

1. An electrosurgical device comprising:
a. a handle portion;
b. an elongate shaft having a proximal end connected to said handle portion and a distal end region comprising a plurality of electrically conductive members including:
i. at least one active electrode having a proximal end and a distal end that is positioned on said elongate shaft at or near the distal end of said elongate shaft,
ii. at least one auxiliary electrode having a proximal portion and a distal portion that is positioned on said elongate shaft in close proximity to said at least one active electrode,
iii. at least one insulating dielectric member disposed between each of said at least one active electrodes and each of said at least one auxiliary electrodes, and
iv. a first conductor connected to said at least one active electrode,
wherein:
the distal portion of said at least one auxiliary electrode is positioned in close proximity to one end of the at least one active electrode so as to increase the energy density in the region surrounding the active electrode;
each of said at least one active electrodes is configured for uninterrupted electrical connection to a power source via said first conductor; and
each of said at least one auxiliary electrodes is electrically connected to said first conductor via circuitry that decreases the power delivered to said at least one auxiliary electrode relative to said at least one active electrode.

2. The electrosurgical device of claim 1 wherein said circuitry contains at least one resistor.

3. The electrosurgical device of claim 2 wherein said resistor has a value between 20 Ohms and 100 mega Ohms.

4. The electrosurgical device of claim 2 wherein said resistor has a value between 300 Ohms and 5 kOhms.

5. The electrosurgical device of claim 2 wherein said resistor has a value between 500 Ohms and 3 kOhms.

6. The electrosurgical system of claim 1 wherein said electrosurgical device further comprises a return electrode.

7. The electrosurgical device of claim 6 wherein said return electrode comprises a ring electrode positioned on said elongate shaft.

8. The electrosurgical device of claim 6 wherein said return electrode is configured for remote mounting to a patient.

9. An electrosurgical system comprising the electrosurgical device of claim 1 in combination with an electrosurgical generator, wherein said electrosurgical generator houses said power source to which each of said at least one active electrodes is electrically connected, further wherein said electrosurgical generator comprises a source of radio frequency energy and first output for connection to said at least one active electrode via cabling and connectors.

10. The electrosurgical system of claim 9 wherein said circuitry connecting each of said at least one auxiliary electrodes to said first conductor is disposed within the electrosurgical device.

11. The electrosurgical system of claim 9 wherein said circuitry connecting each of said at least one auxiliary electrodes to said first conductor is disposed within the electrosurgical generator.

12. The electrosurgical system of claim 9 wherein said circuitry connecting each of said at least one auxiliary electrodes to said first conductor is disposed within said cabling.

13. The electrosurgical system of claim 9 wherein said circuitry connecting each of said at least one auxiliary electrodes to said first conductor is disposed within said connector.

14. The electrosurgical system of claim 9 wherein said circuitry connecting each of said at least one auxiliary electrodes to said first conductor is located within an adaptor external to said generator that serves to connect said electrosurgical device to said first output.

15. An electrosurgical device comprising:
a. a handle portion;
b. an elongate shaft having a proximal end connected to said handle portion and a distal end region comprising a plurality of conductive members including:
i. at least one active electrode having a proximal end and a distal end that is positioned on said elongate shaft at or near the distal end of said shaft,
ii. at least one auxiliary electrode having a proximal portion and a distal portion that is positioned on said elongate shaft in close proximity to said at least one active electrode,
iii. at least one insulating dielectric member disposed between each of said at least one active electrodes and each of said at least one auxiliary electrodes,
iv. a return electrode; and
v. a first conductor connected to said at least one active electrode and a second conductor connected to said at least one return electrode,
wherein:
the distal portion of said at least one auxiliary electrode is positioned in close proximity to one end of the at least one active electrode so as to increase the energy density in the region surrounding the active electrode;
each of said at least one active electrodes is configured for uninterrupted electrical connection to said power source via said first conductor; and
each of said at least one auxiliary electrodes is electrically connected to said second conductor via circuitry.

16. The electrosurgical device of claim 15 wherein said return electrode comprises a ring electrode positioned on said elongate shaft.

17. The electrosurgical device of claim 15 wherein said circuitry includes at least one resistor.

18. The electrosurgical device of claim 17 wherein the value of said resistor is between 0.1 Ohm and 2 kOhms.

19. The electrosurgical device of claim 17 wherein the value of said at least one resistor is between 0.1 Ohm and 100 Ohms.

20. The electrosurgical device of claim 17 wherein the value of said at least one resistor is between 0.1 Ohm and 20 Ohms.

21. An electrosurgical system comprising the electrosurgical device of claim 15 in combination with an electrosurgical generator, wherein said electrosurgical generator houses said power source to which each of said at least one active electrodes is electrically connected, further wherein said electrosurgical generator comprises a source of radio frequency energy and first and second outputs for connection to said at least one active electrode and said return electrode, respectively, via respective cabling and connectors.

22. The electrosurgical system of claim 21 wherein said circuitry connecting each of said at least one auxiliary electrodes to said second conductor is disposed within the electrosurgical device.

23. The electrosurgical system of claim 21 wherein said circuitry connecting each of said at least one auxiliary electrodes to said second conductor is disposed within the electrosurgical generator.

24. The electrosurgical system of claim 21 wherein said circuitry connecting each of said at least one auxiliary electrodes to said second conductor is disposed within said cabling.

25. The electrosurgical system of claim 21 wherein said circuitry connecting each of said at least one auxiliary electrodes to said second conductor is disposed within said connectors.

26. The electrosurgical system of claim 21 wherein said circuitry connecting each of said at least one auxiliary electrodes to said second conductor is located within an adaptor external to said generator that serves to connect said electrosurgical device to said first and second outputs.
